(19) United States
(12) Patent Application Publication
(10) Pub. No.: US 2006/0241521 A1
(43) Pub. Date: Oct. 26, 2006

(54) System for automatic structured analysis of body activities

(76) Inventor: David Cohen, Tel-Aviv (IL)

Correspondence Address: MARTIN MOYNIHAN;c/o ANTHONY CASTORINA, SUITE 207, 2001 JEFFERSON DAVIS HIGHWAY, ARLINGTON, VA, 22202, US

(21) Appl. No.: 11/109,705

(22) Filed: Apr. 20, 2005

(51) Int. Cl. A61B 5/103

(52) U.S. Cl. 600/595

(57) ABSTRACT

A personal emergency response system employs a structured terminology of body activities. Measurements of primary body activities using accelerometers, heart-bit monitors, etc. are converted to secondary and tertiary level body activities such as walk and fall, further sequenced and combined to determine a personal condition such as walk, stumble and fall, and to identify sequences of such conditions. The structured terminology enables a language supporting a functional description of body activities associated with physical and physiological measurements and enables a machine to understand physical activities.

### FIELD AND BACKGROUND OF THE INVENTION

The present invention relates to system and methods providing automatic descriptions and analysis of human activities.

Such a system is useful for machine understanding of situations associated with human, or for that matter animal, activities, more particularly, but not exclusively to system and methods for personal emergency response and social alarms and also to machine description of such activities and the use by the machine of such descriptions in virtual reality type simulations and the like.

The timely identification of a personal emergency situation is important and not a trivial task. Security personnel including night watchman and guards, airline pilots, truck and van drivers and the like can be the subject of attacks and other emergencies with which they are unable to cope. In such a case it is desirable for the subject of the attack to call for help, but sometimes the nature of the emergency renders calling for help impossible. Likewise, elderly and other vulnerable persons, particularly those living on their own, can find themselves in difficulties and unable to reach a telephone to call for help, for example after a fall.

In cases where it is not possible to call for help, a number of systems exist for automatically determining that an emergency situation exists and calling for help.

Hospital-based systems that monitor a patient's pulse and call a doctor or nurse if the pulse falls are well known but are not suitable for anything other than the hospital environment.

Aircraft based hijack warning systems rely upon the pilot's standard radio-based voice link to air traffic control or include panic buttons for broadcasting an SOS signal. Hijackers however tend to be familiar with the presence of these systems and either use them to their advantage or prevent their use altogether.

Other systems for protecting aircraft from emergencies tend to rely on pilots' reaction times. Certain types of emergencies happen too quickly for the pilots to be able to raise the alarm or divert the pilots to emergency activity without diverting their attention to raising the alarm.

Normal activities are different for an old person, a sick person, a disabled person etc., therefore the relevant abnormal activity is also different. Other people may intentionally assume activities that cause substantial physiological stress, which should be considered normal, such as police officers, firefighters, etc. Other people that should be monitored for abnormal situation, where the definition of abnormality may be complex, are people engaged in certain sport activities, people handling hazardous materials, security officers, pilots, etc. The change of the physiological activities that should determine an emergency situation is different for each of these occupations.

Israel Patent Application No. 145498 discloses a system for detecting cockpit emergencies comprising the following:

a) an input unit for receiving body stress level information from at least two subjects,

b) a detection unit, associated with said input unit, for comparing stress level information from said at least two subjects, to detect substantially simultaneous stress level increases in said subjects,

the system being operable to threshold detected simultaneous stress level increases to infer the presence of an emergency situation and to enter an alarm state.

The system uses the physiological state of the pilots to determine that an emergency situation has arisen. In order to reduce false alarms it takes data from the two pilots and deduces the presence of an alarm when both pilots indicate stress. Such a system has the disadvantage that it is only useful in situations such as the cockpit of a civil aircraft where two or more persons are likely to undergo the same emergency. The system is not applicable to security guards, elderly people living alone and the like. Likewise it is not applicable for monitoring of persons being sent into dangerous situations such as troops into battle or firemen into a burning building.

Body language and body activities provide a language that is readily understandable by human beings. However machine processing is currently unable to have even the most basic understanding of physical human activities. It is possible to measure individual movements, but an understanding of concepts such as walking and running is not readily derivable from individual measurements. Rather such concepts arise from an amalgamation of different primary movements. A proper machine understanding of physical human activities would allow machines to better interact with humans, to understand what is happening with them and to be able to simulate humans more realistically

There is a known device that comprises a mercury switch that can be worn on the body, and which issues a signal or alarm when the person reaches a given inclination angle. A problem with this type of device is that it is incapable of distinguishing between a person knocked down in an accident and a person tying his shoelaces.

A flier device, placed on the hand, measures acceleration and angle, and directly sets an alarm based on thresholding of these two measurements. The device is therefore unable to distinguish between a user falling over and for example the user banging his arm on the table and subsequently raising his arm.

Neither of these devices ever attempts to understand the general body context within an overall situation which may be highly complex, but merely automatically sets an alarm. Hence, the vast majority of alarm events are therefore false alarms and are habitually ignored and thus rendered useless.

It is therefore beneficial for a machine to understand complex body activities It is an aim of the present invention to provide a human-machine interface which is able to overcome the above-outlined problem and to understand body activities of a human or other animal.

### SUMMARY OF THE INVENTION

Unless otherwise defined, all technical and scientific terms used herein have the same meaning as commonly understood by one of ordinary skill in the art to which this invention belongs. The materials, methods, and examples provided herein are illustrative only and not intended to be limiting.

Implementation of the method and system of the present invention involves performing or completing certain selected tasks or steps manually, automatically, or a combination thereof Moreover, according to actual instrumentation and equipment of preferred embodiments of the method and system of the present invention, several selected steps could be implemented by hardware or by software on any operating system of any firmware or a combination thereof For example, as hardware, selected steps of the invention could be implemented as a chip or a circuit. As sole, selected steps of the invention could be implemented as a plurality of software instructions being executed by a computer using any suitable operating system. In any case, selected steps of the method and system of the invention could be described as being performed by a data processor, such as a computing platform for executing a plurality of instructions.

According to a preferred embodiment of the present invention there is provided a system for digital processing of body activities, containing: an input for inputting measurements of primary body activities; a prey processing unit for combining the primary body activities into phrases to describe secondary body activities, and a secondary processing unit for combining the phrases into sentences to describe tertiary body activities, the phrases and sentences allowing a digital system to interact with at least the secondary body activities via sensing of the primary body activities.

Also according to a preferred embodiment of the present invention there is provided a system for processing a structure of terms describing body activities, the structure of terms containing: a set of primary terms, each the primary term describing a measurement of a body activity a, set of combined terms, each the combined term describing a body activity, each the combined tern containing at least one of a concurrent combination, a sequential combination and a temporal combination, of at least one of the primary terms, other the combined terms, and time measurements.

Further according to another preferred embodiment of the present invention there is provided a computer executable software program to interactively create a structure of terms, the structure of terms containing: a set of primary terms, each the primary term describing a measurement of a body activity, a set of combined terms, each the combined term describing a body activity, each the combined term containing at least one of a concurrent combination, a sequential combination and a temporal combination, of at least one of the primary terms, other the combined terms, and time measurements.

Still fisher according to another preferred embodiment of the present invention there is provided a computer executable software language useful to define rules, the rules operative to identify to an electronic system situations demanding response, the language constructed of terms describing body activities, the terms constructed of at least one of the terms, measurements of body activities, time measurements and sequences thereof.

According to yet another preferred embodiment of the present invention there is provided a computer executable software program to interactively define rules, the rules operative to identify to an electronic system situations demanding a response, the language constructed of terms describing body activities, the terms constructed of at least one of the terms, measurements of body activities, time measurements and sequences thereof, and use them to define rules that identify situations of personal emergency.

According to still another preferred embodiment of the present invention there is provided a computer executable software program operative to: interactively create a structure of terms, the structure of terms containing: a set of primary terms, each the primary term describing a measurement of a body activity; a set of combined terms, each the combined term describing a body activity, each the combined term containing at least one of a concurrent combination, a sequential combination and a temporal combination, of at least one of the primary terms, other the combined terms, and time measurements; and interactively use the structure of terns to create at least one sequence of body activities, the sequence operative to perform at least one of animation of a figure on a visual display and operating a robot.

Additionally according to a preferred embodiment of the present invention there is provided a computer executable software program wherein the sequence of body activities describes a situation of a personal emergency.

Also according to a preferred embodiment of the present invention there is provided a structure of terms wherein the measurements of body activities comprise at least one of: a measurement of the acceleration of at least one of a limb or the entire body, a measurement of the velocity of at least one of a limb or the entire body; a measurement of the angular velocity of at least one of a limb or the entire body; a measurement of the orientation of at least one of a limb or the entire body; a measurement of the distance of at least one of a limb or the entire body from a solid surface; a measurement of the distance between at least two limbs; a measurement of the temperature of at least one of a limb or the-entire body, a measurement of the skin conductivity of at least one of a limb or the entire body; a measurement of the heart bit rate; a measurement of respiratory sounds; a measurement of bodily electromagnetic signals.

In this context, measurements are made and then understood in absolute terms or in logical context or in relative context.

Logical context refers to the ability to take into account current circumstances in understanding the measurement. For example, if the subject starts running, then an increase in heart rate is only to be expected, and should not in itself set an alarm.

Relative context refers to two measurements that in themselves may not indicate a problem, but their proximity to other events or perhaps each other indicates that there is a problem. Thus, if a person is completely at rest, but gives a heart rate reading which shows an increase to 110 or more beats per minute, we can infer from the relative context that something is wrong.

Absolute context may be used to refer to problems by comparison with a fixed threshold.

Further according to a preferred embodiment of the present invention there is provided a personal emergency alarm network containing: at least on personal activity monitoring apparatus operative to perform at least one measurement of body activity; an emergency monitoring server, the personal activity monitoring apparatus operative to transmit data to the emergency monitoring server; the apparatus operative to provide at least one first nomenclature for at least one measurement of a body activity surpassing the at least one threshold; provide at least one second nomenclature for at least one a first combination of at least one of a concurrent combination, a sequential combination and a temporal combination, the first combination containing at least one of the body activity and the first nomenclature; provide at least one third nomenclature for at least one a second combination of at least one of a concurrent combination, a sequential combination and a temporal combination, the second combination containing at least one of the body activity, the first nomenclature, the second nomenclature and the third nomenclature; provide definitions of emergency situations; associate the definitions of emergency with at least one of at least one of the body activity, the first nomenclature, the second nomenclature and the third nomenclature, the server being operable to receive and understand said nomenclature. The server may carry out further processing on the data or not as the case may be. That is to say the apparatus may send out nomenclature data for further processing by the remote server. Alternatively the apparatus may send out binary decision data such as “occurrence of the emergency”, nature of the emergency” “location” etc, and simply requiring the remote server to raise the appropriate alarm.

It is preferred to send the location first, so that if further transmissions fail, at least the location at which help is required is known.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in order to provide what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a simplified illustration of a preferred embodiment of the present invention showing a system for automatic structured analysis of body activities;

FIG. 2 is a simplified illustration of a system for automatic structured analysis of body activities according to another preferred embodiment of the present invention;

FIG. 3 is a simplified illustration of a system for monitoring personal emergency situations according to a preferred embodiment of the present invention;

FIG. 4 is a simplified illustration of a system for measuring body activities in accordance with the system for monitoring personal emergency situations of FIG. 3;

FIG. 5 is a simplified illustration of a preferred structure of body activities useful to interpret the measurements of body activities of FIG. 4 and in accordance with the system for monitoring personal emergency situations of FIG. 3;

FIG. 6 is a simplified illustration of a structure of processing steps for interpreting the measurements taken by the system of FIG. 4, according to the structure of FIG. 5, and in accordance with the system for monitoring penal emergency situations of FIG. 3.

FIG. 7 is an illustration of a computer display of a preferred embodiment of the present invention.

### DESCRIPTION OF THE PREFERRED EMBODIMENTS

The principles and operation of a system for automatic analysis and visualization of human activities according to the present invention may be better understood with reference to the drawings and accompanying description. The present invention provides a hierarchical system within which human or for that matter animal physical and/or physiological behavior can be analyzed in a way that is understandable to the digital world.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

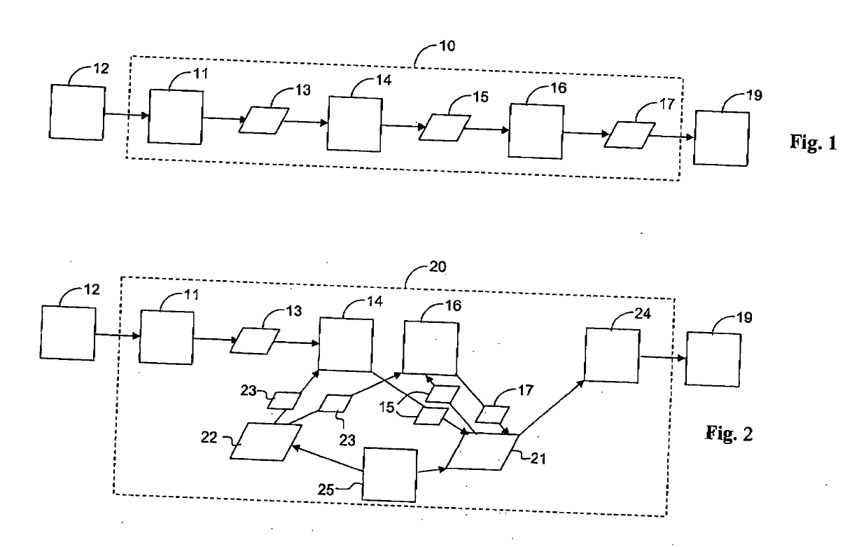

Reference is now made to FIG. 1, which is a simplified illustration of a preferred embodiment of the present invention showing a system for automatic structured analysis of body activities 10. The system 10 comprises the following elements:

an input device 11 which inputs signals, say from a sensor 12. The sensor 12 is operative to sense at least one body activity such as angle, velocity, acceleration, heart beat, skin conductivity, etc. The input device 11 process the signals received from the sensor 12 and outputs the signal in a digital form 13 acceptable for processing by a computing device such as a micro-controller, a computer, etc. The sensor is preferably a single unit mounted on the trunk part of the body of the user,

a primary processing unit 14 operative to process the measurement 13 and create a “phrase” 15 that describes a secondary body activity, which is typically and preferably a concurrent or a sequential or a temporal combination of at least one type of measurements 13, or a combination of such sequences;

a secondary processing unit 16 operative to process the measurement 15 and create a “sentence” 17 that describes a tertiary body activity, which is typically and preferably a concurrent or a sequential or a temporal combination of at least one type of phrase 15, or a combination of such sequences. The “sentence” 18 may for example allow a digital system 19 to determine whether a phrase or a sentence or one of their combinations is an emergency situation and act accordingly.

It will be appreciated that the processing may be carried out locally at the measurement site, that is at or near the person or persons who are the subject of the measurements, or the measurements may be transmitted and the processing be carried out remotely. An advantage of carrying out processing at or near the subject is that the transmission bandwidth is reduced since only processing results of the measurement at one level or another are transmitted. The advantage of carrying out processing remotely is that fewer computing resources are needed at the person being measured.

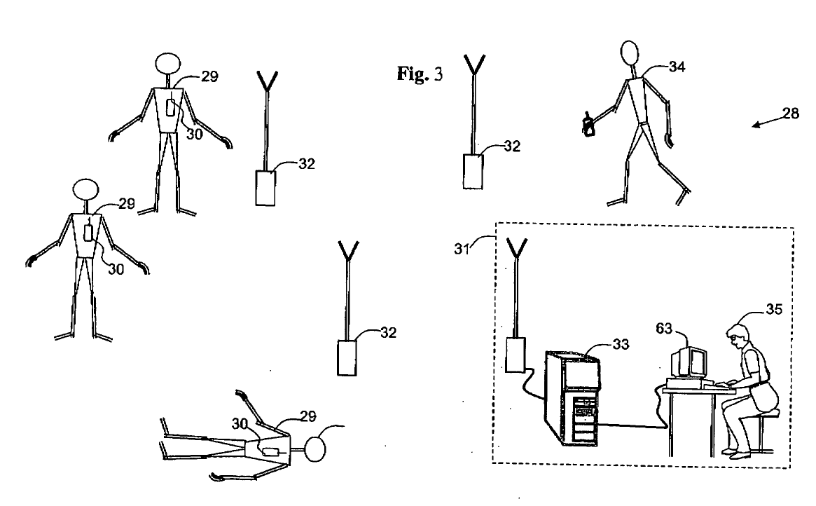

Reference is now made to FIG. 2, which is a simplified illustration of a another preferred embodiment of the present invention showing a system for automatic structured analysis of body activities 20. The system 20 comprises the following elements:

The input device 11 operative to receive input signals from at least one sensor 12. The sensor 12 is operative to sense at least one body activity such as heart bit, skin conductivity, acceleration, etc. The input device 11 process the signals received from the sensor 12 and outputs the signal in a digital form 13 acceptable for processing by a computing device such as a micro-controller, a computer, etc.;

a primary processing unit 14 is operative to process the measurement 13 and create the “Phrase” 15 and store it in a pool 21. The primary processing unit 14 performs the processing using a pool 22 of rules 23. Each such rule, typically and preferably, is a concurrent or a sequential or a temporal combination of at least one type of measurements 13, or a combination of such sequences;

the secondary processing unit 16 is operative to process the “phrases” 15 and create the “sentence” 17 that describes a tertiary body activity, which is typically and preferably a concurrent or a sequential or a temporal combination of at least one type of phrase 15, or a combination of such sequences;

an interface unit 24 enables the digital system 19 to retrieve the phrases and sentences and determine whether a phrase or a sentence or one of their combinations is an emergency situation, and act accordingly.

A user interface module 25 enables a user to manage the storage pools 26 and 21 and to define the rules 27.

Reference is now made to FIG. 3, which is a simplified illustration of a preferred embodiment of the present invention showing a system for monitoring personal emergency situations 28. The system 28 monitors subjects 29, who are typically individuals that may encounter situations that require immediate help. Typically such individuals may be, but are not limited to old people fail people, disabled people, sick people or people otherwise in a dire medical situation, people with limited or disturbed cognitive abilities or other mentally challenged people, etc. Alternatively and additionally, such individuals may be, but are not limited to people in hazardous occupations, such as police officers, firefighters, security officers, people handling hazardous materials, soldier on duty, etc. Further alternatively and additionally, such individuals may be people operating in remotely or alone, such as truck drivers, people engaged in outdoors sport activity, etc. Further alternatively and additionally, such individuals may be people operating in secluded places such as aircraft pilots, train drivers, etc. All these people and others can usefully be provided with continuous monitoring to assess their situation to determine whether they are in need of immediate help, and, if possible, the cause of the situation and the kind of help needed.

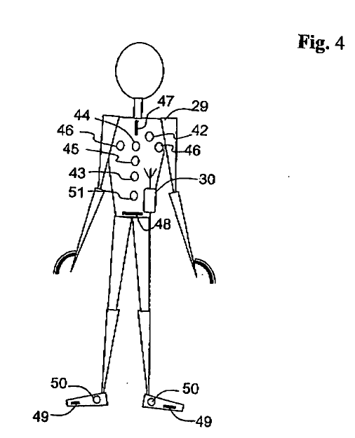

Preferably, the individuals who require monitoring, are continuously monitored for a variety of physical, biological and physiological activities as will be detailed in FIG. 4. These monitored signal are collected by a transceiver 30, that preferably transits the measurements to a monitoring center 31, preferably via a network of relay stations 32, such as a cellular communication network, a trunked radio communication network, a wireless area network such as IEEE 802,16, a local wireless network such as IEEE 802.11, etc.

Preferably, a computer 33 operative in the monitoring center 32, collects the measurements, analyses them, and provides alerts and alarms according to the perceived situation. The alerts and alarms can be transmitted immediately to people 34 who are in charge of the situation, such as fellows, shift managers, commanders, rescue teams, medical teams, etc., or it can be first monitored by an attendant 35, who dispatches the required personnel.

The computer 33 is preferably also operative to store all the collected measurements and retrieve them upon request.

The present embodiments, as shown and described in FIG. 1 and FIG. 2, can be configured in the system for monitoring personal emergency situations 28 in several ways.

In one configuration, following FIG. 1 and FIG. 3, the input device 11 is incorporated in the transceiver 36, while the computer 33 comprises the primary processing unit 14 and the secondary processing unit 16, and optionally the digital system 19.

Alternatively and preferably, the input device 11 and the primary processing unit 14 are incorporated in the transceiver 37, while the computer 33 comprises the secondary processing unit 16 and optionally the digital system 19.

Alternatively and further preferably, the input device 11, the primary processing unit 14 and the secondary processing unit 16 are incorporated in the transceiver 38, while the computer 33 comprises the digital system 19.

Also alternatively, following FIG. 2 and FIG. 3, the input device 11 is incorporated in the transceiver 39, while the computer 33 comprises the primary and the secondary processing units 14 and 16, the pools 22 and 21, the interface unit 24 and the user interface unit 25, and optionally the digital system 19.

Alternatively and preferably, the input device 11, the primary processing unit 14, and a mirror copy of the pool 22 are incorporated in the transceiver 40, while the computer 33 comprises the secondary processing units 16, the pools 22 and 21, the interface unit 24 and the user interface unit 25, and optionally the digital system 19.

Alternatively and Other preferably, the input device 11, the primary processing unit 14 the secondary processing units 16 and a mirror copy of the pools 22 and 21 are incorporated in the transceiver 41, while the computer 33 comprises the pools 22 and 21, the interface unit 24 and the user interface unit 25, and optionally the digital system 19.

Reference is now made to FIG. 4, which is a simplified illustration of a preferred embodiment of the present invention showing a system for measuring body activities. FIG. 4 shows a monitored subject 29 equipped with several measuring devices, each device is capable of measuring at least one biophysical phenomenon. The devices communicate with the transceiver 30 via wire or wireless technologies. Some of the devices may provide analog output that is digitized by the transceiver 30, some devices may digitize the measurement and provide digitized output, for example via USB protocol, some devices may digitize the measurement and provide digitized output by means of wireless communications such as IEEE 802.15.1, IEEE 802.15.3, or 802.15.4.

In a preferred embodiment of the present invention, a device 42 measures the heart beat, a device 43 measures the body temperature, a device 44 measures sweat, for example by measuring the conductivity of the skin, a device 45 measures respiratory sounds, devices 46 measures electromagnetic signals of the body, such as ECG, a device 47 measures the vertical orientation (or tilt) of the torso, a device 48 measures the horizontal orientation (or tilt) of the hips, the devices 49 measures the acceleration of a body limb, in this example by measuring the acceleration of each shoe, the devices 50 measures the distance between two limbs, in this example by measuring the distance between themselves, a device 51 measures the distance of at least one of a limb, the torso in this case, from a solid surface.

Transceiver 30 collects the signals provided by the measuring devices and transmits them to the monitoring center 31. The monitoring center 31 may be located within a short distance, such as when monitoring the activities of firefighters from a near by command and control car, or remotely, such as when monitoring soldiers or frail people at their homes. Transceiver 30 may also comprise a positioning device, such as of a global positioning system (OPS), to report its position to the monitoring center 3.

The transceiver 30 preferably transmits the measurements to the monitoring center 31 as the measurements are provided by the measuring devices. Alternatively and preferably the transceiver 30 transmits only measurements that differ from a predefined value, or from the preceding value, by a specific threshold value. Also alternatively and preferably the transceiver 30 collects the measurements and transmits them in packets at specific time intervals. Further alternatively and preferably the transceiver 30 performs some processing on at least some of the signals, preferably on the acceleration measurements and transmits only the results of the processing. For example, the transmitter 12 processes the respiratory sounds and transmits the resulting rate instead of the sound. Even further alternatively, the transmitter 12 is operative to receive commands from the monitoring center 31 and transmit the original measurements of a specific body activity in real-time. In a yet further embodiment the processing to be described below is carried out at the user and higher level derivations of the measurements are transmitted. Such a further embodiment is particularly advantageous as it leads to major reductions in bandwidth usage.

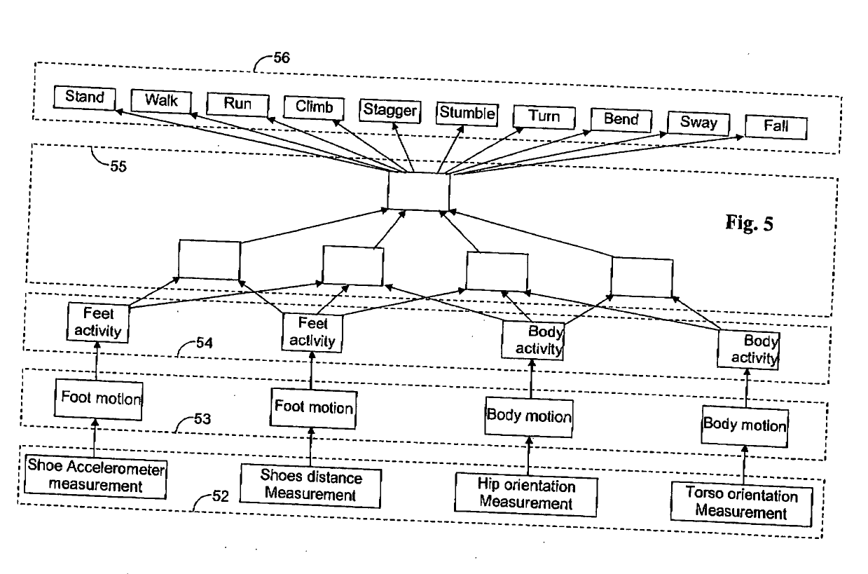

Reference is now made to FIG. 5, which is a simplified illustration of a preferred embodiment of the present invention showing a structure of terms describing body activities.

At the bottom line of FIG. 5 there are first-level measurements 52 of body activities preferably received from respective measuring devices, such as: heart beat rate, body temperature, skin conductivity, respiratory sounds, electromagnetic signals, vertical orientation, horizontal orientation, acceleration of a body limb, velocity of a body limb, the distance between two limbs, the distance of the body from a solid surface, etc.

Preferably, these first-level measurements 52 are integrated, differentiated or otherwise calculated to provide second-level measurements 53 of body activities, such as calculating speed from acceleration and calculating rate from a sequence of single heart beat measurements. For example, orientation angle may be continuously measured, and then a regular change in orientation angle may be interpreted as a sway, whereas a continuously held orientation angle may be interpreted as a tilt.

The first and second level measurements of body activities are then preferably processed to provide third-level measurements 54 of body activities. For example, a certain sequence of measurements of the acceleration of the shoes, together with a sway, indicates a walk at a certain speed, or climbing a staircase, or staggering. Likewise, a certain sequence of measurements of the distance between the shoes, preferably together with a given sway, also indicates a walk at a certain speed. A certain sequence of measurements of the orientation of the hips, preferably again combined with a sway, also indicates a walk at a certain speed.

Acceleration beyond a certain threshold, together with impact-type sounds, can be interpreted as a shock, for example as a result of being hit. Sounds can also be analyzed for meaning, and then understood with or without context. For example the subject may call out “help”, which should automatically set up an alarm state. If the term is accompanied by a significant change in heart rate or respiratory rate then it is clear that something has happened.

Similarly, orientation angles of the body or a limb can be continuously measured and when the angle surpasses at least one of predefined thresholds, or when the rate of change of the angle surpasses at least one of predefined thresholds, a third level deduction of falling may be the result

Combinations of specific lower level measurements are also preferably processed to provide forth level indications 55. Fourth level indications combine the third level indications to understand behavior, thus a run followed by falling followed by impact followed by lying on the floor may indicate an accident, whereas a run followed by falling followed by impact followed by lying on the floor followed by a further impact may suggest that the person being monitored is under attack.

Typically at least some of the second, third and fourth levels of measurements of body activities preferably involve time measurements that are acquired from a clock, or from timers calculating elapsed time between specific measurements, or lack of such.

Fourth, third, second and first body activities, as well as time measurements, are then preferably combined, sequenced, processed and compared at an even higher level to determine one of a fifth level of body activities 56, which is the assumed bodily condition or activity of the subject.

The Fourth, third and second body activities typically and preferably form the phrases 15 of FIGS. 1 and 2, while the fifth level of the body activities typically and preferably form the sentences 17 of FIGS. 1 and 2.

That is to say, individual primary measurements are formed in the second level to form words that describe activity. At the third level these words combine to form phrases and at the fourth and fifth level, super-phrases or sentences are generated.

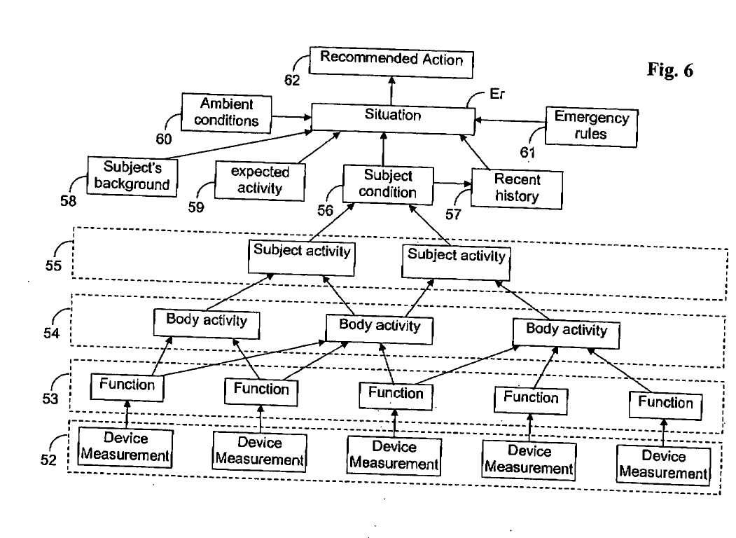

Reference is now made to FIG. 6, which is a simplified illustration of a preferred embodiment of the present invention showing a processing steps for interpreting the measurements taken by the system of FIG. 4, according to the structure of FIG. 5, and in accordance with the system for monitoring personal emergency situations of FIG. 3.

The structure of processing steps preferably comprises the processing of the first level 52, second level 53, third level 54, fourth level 55 and fifth level 56 of body activities described with reference to FIG. 5. The body activity of the highest level, preferably level five in this example, is then added to the recent history 57 of events occuring to the subject, compared with the subject's background 58, the subject's expected activity 59 and the ambient condition 60 to determine, according to a pool of rules 61 how the situation is to be understood. A recommended reaction is made to the user, or an action 62 is then provided to an attendant or emergency crew or any other person who is in charge, or responsible.

The rule base 61 is a collection of assumptions of situations that pertain to the activity of the user, whether regular activities, abnormalities or emergencies. Such assumptions may depend on the subject's condition, environment, situation, etc. For example, for an old person, certain types of unsteady movement which would look highly unusual in a fit person would not be considered abnormal. Likewise an adult with a sedentary occupation who suddenly starts running may be assumed to be be in danger, whereas for a child, running this way is not unusual. A staggering firefighter would be expected to require assistance. A soldier or a sportsman falling would not be considered abnormal. However, a frail person falling would be suspected to be in a state of emergency. A policeman on a routine patrol encountering a shock may be in a state of emergency while a policeman controlling a riot is considered to need assistance if he is noted as being hit, falling and then being hit again or not showing vital signs.

The rules are expressed using a terms or labels built into a language comprising the structure of human and body activities terms as described above. For example, emergency situations can be expressed as:

BEND and STRAIGHTEN=IGNORE

IMPACT and BEND and STRAIGHTEN and IMPACT=ALARM

OLD MAN and STAGGER and AT LEAST 10 SECONDS and FALL TO MORE THAN 55 DEGREES and LAY STILL and OVER 2 MINUTES=ALARM

POLICEMAN and riot and STAGGER and IMPACT and FALL and RISE and WALK=IGNORE

POLICEMAN and riot and STAGGER and IMPACT and FALL and REPEATED IMPACT FOR OVER 30 SECONDS=SEVERE ALARM

The above two cases make the point that relatively subtle differences the order of events can give rise to completely different outcomes. Such differences are very clear to humans but have up till now caused difficulty for digital systems. The use of the present embodiments thus provides machine processing with a natural basis on which to understand these subtleties. These variations allow for suitable programing to be used for different policemen in different circumstances or operations. In the second case it is apparent that the policeman is on the floor and being kicked.

Preferably there may be many such rules that apply to a specific subject. The computer 33, continuously processes the recent events to check for a possible match to at least one rule. It is also possible that more than one rule is fulfilled at a certain point of time. It is further possible that a short time after one rule is fulfilled another rule is also fulfilled. In certain cases such situation may lead to an alleviated state of emergency, while in other situations the state of the emergency may be demoted.

It is appreciated that the analysis of several combinations of measurements and sequences of measurements can lead to different conclusions. The computer 33 is operative to resolve such situations and determine a prevailing situation based on statistics, fuzzy logic and other adequate mathematical methods.

In some cases contradicting body activities preferably result in rejection of a measurement, such as rejecting null heart bit rate if the subject is walking steadily. On the other hand, contradicting body activities preferably may be interpreted as a suspected emergency, for example if the breath rate and heart bit rate increase when the subject is still.

Combinations and sequences of body activities are then observed to determine the state of emergency and suggest an appropriate response. If the situation requires so, an alert is provided to the attendant 35 or directly to the rescue team 34 of FIG. 3.

Preferably, some of the processing and conclusions associated with the second, third, fourth and fifth body activities are provided by the transmitter 30 to reduce the amount of transmissions, save bandwidth and save battery power.

The computer 33 is preferably operative to retrieve the stored measurements and display them, preferably in the order in which they occur, preferably at any required level of body activity.

In one preferred embodiment, the computer 33 is preferably operative to use the words, phrases and sentences to animate the activity of a subject, simulating the subject's behavior and motions, preferably at the rate in which they occur, alternatively and preferably at a faster rate. The computer 33 receives the words, phrases or sentences from the subject and applies them to a virtual subject on screen which then carries out the activities indicated by the words, phrases and sentences. For example, when the term “walk” is received, the virtual subject walks. The system is preferably operative to provide the exact location and posture of the subject. Preferably, if a three dimensional model of the environment is available, the computer 33 is able to display the location, activity and posture of the subject within the environment.

In a further preferred embodiment, no measurements arc taken. Rather the words, phrases and sentences are put together by a programmer or compiled automatically from a program, and applied to the virtual subject. Thus it is possible to use the hierarchy of words, phrases and sentences to define behaviors of viral actors.

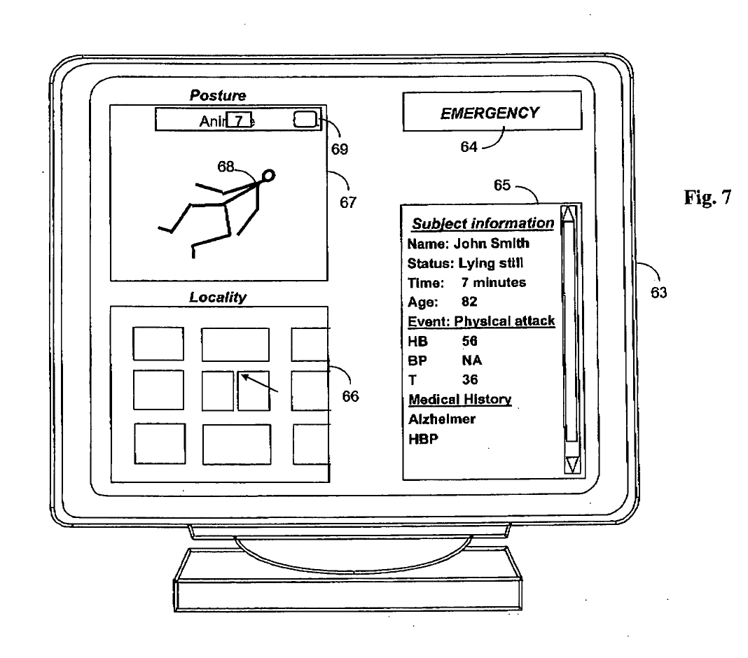

Reference is now made to FIG. 7, which is an illustration of a computer display of a preferred embodiment of the present invention, preferably a monitor 63 of the computer 33 as shown in FIG. 3. The monitor 63 preferably displays the status of the situation 64 as “emergency”, the details of the subject 65 and the real-time values of selected relevant measurements of body activities, the locality of the event 66, based on GPS, GIS and other information acquired from sources of three-dimensional modeling, and the posture 67 of the subject. Preferably, the user of the monitor 63 can animate the FIG. 68 by selecting the time and depressing a button 69.

The present embodiments can be preferably used to animate any object for which a structure of phrases and sentences has been collected and arranged. In a preferred embodiment of the present invention the user may construct sentences made of sequences of phrases, each of which is in itself a combination of lower level phrases. The higher level words and phrases allow the user to avoid having to specify body activities at a low level, as present day animators are required to do. The computer 33 then processes the sentences into their phrases and the phrases into their lower level terms and display on the computer's screen the temporal behavior of the subject and each of his body parts according to the contents of the preferred structure of terms.

The processing of the measurements and body activities to provide higher-level body activities is preferably performed in a manner that enables replacement and improvements of lower level functions. Thus, if a certain state of emergency, is determined based on a certain sequence or combination of events, such as a walk, shock and fall, the measurement or the processing that determines the walk, or shock, of fall can be replaced or improved at a lower level, without affecting the upper level. For example, the upper level is not affected if the subject is equipped with accelerometers 49 in the shoes to determine the walking activity, or distance sensors 50, or hip orientation sensor 48. Furthermore, low level definitions can be set differently for different people without effecting upper level decision-making. Thus speed and time-based thresholds could be set differently for young people and old people.

Consequently, as the subject performs an action, the system interprets the action in three dimensions in its context, with reference to the subject's background, the subject's duty and the ambient situation.

In one preferred embodiment the present embodiments are provided in combination with a video camera and image processing system. The camera watches a particular user, or an area in which one or more persons are to be found. The image processing system identifies reference points on the body of any person visible and then makes primary measurements based on the reference points. The present embodiments are then able to identify physical activities and thereby understand what is happening in the video image. For example such a system may be able to distinguish between people dancing (jumping and no impacts) and people fighting (jumping, falling and impacts).

The system enables a user to define structured terminology of human activities, based on interpretations of body activities that are based on interpretations of physiological measurements. Such terminology may be

Example of a basic physical measurements:

LEVEL 1

1. Measure body recline and limbs orientation in three dimensions angles.

LEVEL 2

2. Calculate change of recline and orientation as a function of time.

3. Calculate angles as directional acceleration and velocity

4. Compare values with predefined thresholds, determine MOTION, IMPACT, etc,

LEVEL 3

5. Integrate with other measurements such as the motion of other limbs, height from the ground, etc.

6. Determine RECLINE, TURN, TILT, SWAY, etc.

LEVEL 4

7. Analyze the probable cause for the motion, such as intentional or external.

8. Analyze in the context of previous measurements and analysis.

9. Determine, SIT, LAY-DOWN, INTENTIONAL-FALL, UN INTENTIONAL-FALL, KNOCKED-DOWN, WALK etc

LEVEL 5

10. Analyze with respect to the precondition of the monitored subject and the situation, determine emergency situation.

Measurements of Motion and Their Logical Assumptions,

1. Motion

2. Step count

3. Directional impact as value, e.g. impact of 2 g

4. Directional impact by logical pattern, e.g. impact relative to object

5. Impact in logical context, e.g. police officer patrolling a hostile neighborhood.

6. Impact by relative context, e.g. an impact of 4 g means someone clubbed the police officer.

GPS or Other Location System gives Absolute Positioning

1. Location as value, e.g. is the subject where the subject is supposed to be?

2. Location by logical pattern, e.g. following an expected path.

3. Location in logical context, how long is the subject in given position at given time.

7. Location by relative context, where is the subject is relative to object.

1. Relative positioning, the location of the subject relative to the location of his equipment.

1. Location as a directional value, e.g. 30 degrees south of the post.

2. Directional by logical pattern, e.g. walking around the post.

3. Direction in logical context, e.g. two police officers going separate ways.

4. Direction by relative context, e.g. two police officers leaving the patrol car in separate ways.

Time:

Time is connected to all other events. Each event receives a different value according to the duration of the event and the timing with respect to other events.

1. Absolute time, needed to decide that something is what should be happening at this time. He is supposed to move at 11 AM

2. Relative time, measure time that the subject runs, running a few seconds is OK, but if the subject runs too long perhaps the subject is running away from something.

3. Sequence of events in time frame.

Body or Physiological Events.

Pulse, breathing, sweat, change in physical attributes,

1. Absolute value, e.g. heart bit rate=70→NORMAL

2. Relative value, change, e.g. heart bit rate increased by 20%→NORMAL CHANGE OF POSTURE

3. As a part of logical pattern, e.g. RISING

4. logical context, e.g. RISING FROM HIS SEAT

5. relative context, e.g. CAR DOOR OPENED

Physical attributes

Is the subject running, jumping, sleeping, sitting, etc.

Impact assessment derived from measurements of acceleration, which can be measured as linear acceleration and as angular acceleration.

1. Impact value

2. Absolute value, unrelated and unassociated (yet)

3. Directional: comes from behind, comes from in front, comes from right, comes form left, comes from above, comes from below

4. Relative, assumed object or person as a cause for the impact

5. As part of logical pattern, e.g. a sequence of impacts

6. In its logical context, e.g. stagger, fall

7. In its relative context, e.g. police officer in a riot.

Example of Continuous Monitoring of a Subject

The subject is a security officer on guards The subject task is to stand in position and check passers by.

Body angle>85→STANDING STRAIGHT, status→OK;

Body motion detected<1 meter/second, body angle>85, status=OK;

High acceleration detected for a short tinge;

Body accelerating forward;

Body angle<80;

Assumed impact from behind, >1.5 g.

Body forward acceleration grows;

Body angle<60;

Second impact detected

Emergency Situation determined

Body forward acceleration grows;

Body angle<60;

Third impact detected from the front.

Body movement not detected.

Emergency Situation elevated.

Typical Expressions using the Aforementioned Language and Terminology:

BEND and STRAIGHTEN and IMPACT=IGNORE

IMPACT and BEND and STRAIGHTEN and IMPACT=ALARM

The advantages of the presently preferred embodiments are:

Saving time and reducing errors by not having to retype long sequences again and again.

Readability, enabling an application programmer to use natural language that pertains to human behavior rather than have to use or decipher the physical meaning of long sequences of obscure physiological measurements.

Usability, as the user can use plain language terms rather than professional physiological terms.

Upgradeability, when you can improve a lower level term (sequence) by adding a measurement or modifying a threshold, that will automatically affect all the higher level terms and rules.

It is expected that during the life of this patent many relevant devices and systems will be developed and the scope of the terms herein is intended to include all such new technologies a prior.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

What is claimed is:

1. A system for digital processing of body activities, comprising:
an input for inputting measurements of primary body activities;
a primary processing unit for combining said primary body activities into phases to describe secondary body activities, and
a secondary processing unit for combining said phrases into sentences to describe tertiary body activities, said phrases and sentences allowing a digital system to interact with at least said secondary body activities via sensing of said primary body activities.

2. A system for processing a structure of terms describing body activities, said structure of terms comprising:
A set of primary terns, each said primary term describing a measurement of a body activity;
A set of combined terms, each said combined term describing a body activity, each said combined term comprising at least one of a concurrent combination, a sequential combination and a temporal combination, of at least one of said primary terms, other said combined terms, and time measurements.

3. A computer executable software program to interactively create a structure of terms, said structure of terms comprising:
A set of primary terms, each said primary term describing a measurement of a body activity;
A set of combined terms, each said combined term describing a body activity, each said combined term comprising at least one of a concurrent combination, a sequential combination and a temporal combination, of at least one of said primary terms, other said combined terns, and time measurements.

4. A computer executable software language useful to define rules, said rules operative to identify to an electronic system situations demanding response, said language constructed of terms describing body activities, said terms constructed of at least one of said terms, measurements of body activities, time measurements and sequences thereof.

5. A computer executable software program to interactively define rules, said rules operative to identify to an electronic system situations demanding a response, said language constructed of terms describing body activities, said terms constructed of at least one of a group comprising: measurements of body activities, time measurements and sequences thereof, and use them to define rules that identify physical behavior.

6. A computer executable software program operative to:
interactively create a structure of terms, said structure of terms comprising:
A set of primary terms, members of said set describing a measurement of a body activity, A set of combined terms, each said combined term describing a body activity, each said combined term comprising at least one of a concurrent combination, a sequential combination and a temporal combination, of at least one of said primary terns, other said combined terms, and time measurements; and
interactively use said structure of terms to create at least one sequence of body activities, said sequence operative to perform at least one of a group comprising animation of a figure on a visual display and operating a robot.

7. A computer executable software program according to claim 6 wherein said sequence of body activities describes a situation of a personal emergency.

8. A structure of terms according to claim 1 wherein said measurements of body activities comprise at least one of:
a measurement of the acceleration of at least one of a limb or the entire body;
a measurement of the velocity of at least one of a limb or the entire body;
a measurement of the angular velocity of at least one of a limb or the entire body;
a measurement of the orientation of at least one of a limb or the entire body;
a measurement of the distance of at least one of a limb or the entire body from a solid surface;
a measurement of the distance between at least two limbs;
a measurement of the temperature of at least one of a limb or the entire body;
a measurement of the skin conductivity of at least one of a limb or the entire body;
a measurement of the heart bit rate;
a measurement of respiratory sounds;
a measurement of bodily electromagnetic signals;
sound measurements.

9. A method for identifying situations of personal emergency, said method comprising the steps of:
providing at least one measurement of a body activity;
providing at least one threshold for at least one of said measurement of a body activity,
providing at least one first nomenclature for at least one said measurement of a body activity surpassing said at least one threshold;
providing at least one second nomenclature for at least one a first combination of at least one of a concurrent combination, a sequential combination and a temporal combination, said first combination comprising at least one of said body activity and said first nomenclature;
providing at least one third nomenclature for at least one a second combination of at least one of a concurrent combination, a sequential combination and a temporal combination, said second combination comprising at least one of said body activity, said first nomenclature, said second nomenclature and said third nomenclature;
providing definitions of emergency situations;
associating said definitions of emergency with at least one of at least one of said body activity, said first nomenclature, said second nomenclature and said third nomenclature.

10. A method according to claim 9 and wherein said step of providing at least one measurement of a body activity comprises providing at least one of:
a measurement of the acceleration of at least one of a limb and the entire body;
a measurement of the velocity of at least one of a limb and the entire body;
a measurement of the angular velocity of at least one of a limb and the entire body;
a measurement of the orientation of at least one of a limb and the entire body,
a measurement of the distance of at least one of a limb and the entire body from a solid surface;
a measurement of the distance between at least two limb$;
a measurement of the temperature of at least one of a limb and the entire body;
a measurement of the skin conductivity of at least one of a limb and the entire body;
a measurement of the heart rate;
a measurement of respiratory activities; and
a measurement of bodily electromagnetic signals.

11. A method according to claim 9 and additionally comprising the step of providing an alarm associated with an emergency based on at least one of said body activities to at least one remote location.

12. A method according to claim 11 and additionally comprising at least one step of providing to said remote location a visual description of said emergency situation.

13. A method according to claim 12 and wherein the step of providing said visual description comprises providing visualization of at least one of said body activity, said first nomenclature, said second nomenclature and said third nomenclature.

14. A method according to claim 9 and additionally comprising the steps of:
collecting said measurements of body activities;
storing said measurements of body activities;
analyzing measurements of body activities according to said first, second and third nomenclatures;
identifying said emergency associated with said first, second and third nomenclatures;
sending at least one alarm associated with said at least one emergency to at least one remote location.

15. A method according to claim 14 and additionally comprising the step of displaying a visualization of said nomenclature to said remote location.

16. A personal emergency alarm network comprising:
at least on personal activity monitoring apparatus operative to perform at least one measurement of body activity;
an emergency monitoring server,
said personal activity monitoring apparatus operative to transmit said measurement to said emergency monitoring server;
said monitoring apparatus being operative to
provide at least one first nomenclature for at least one said measurement of a body activity surpassing said at least one threshold;
provide at least one second nomenclature for at least one a first combination of at least one of a concurrent combination, a sequential combination and a temporal combination, said first combination comprising at least one of said body activity and said first nomenclature;
provide at least one third nomenclature for at least one a second combination of at least one of a concurrent combinations a sequential combination and a temporal combination, said second combination comprising at least one of said body activity, said first nomenclature, said second nomenclature and said third nomenclature;
provide definitions of emergency situations;
associate said definitions of emergency with at least one of at least one of said body activity, said first nomenclature, said second nomenclature and said third nomenclature, and
send data comprising at least said nomenclature to said emergency monitor server.

17. A method according to claim 16 and additionally comprising the step of providing an alarm associated with said emergency to a remote location.

18. A method according to claim 17 and additionally comprising at least one step of providing said remote location with a visual description of said emergency situation.

19. A method according to claim 18 and wherein the step of providing said visual description comprises providing visualization of at least one of said body activity, said first nomenclature, said second nomenclature and said third nomenclature.

20. A method according to claim 3, further comprising at least one of:
a. measuring a recline angle in three dimensions;
b. measuring a change in recline angle as a function of time;
c. interpreting change in recline angle as a function of time as logical assumptions of physical state;
d. interpreting change in recline angle as a function of time as a logical assumption about a cause of a physical state;
e. defining a directional source of acceleration;
f predicting a sway path from a defined directional source of acceleration; and
g. taking an absolute context of respective measurements.

21. A method according to claim 3, wherein said terms comprise at least one of a group comprising: motion, step count, directional impact as value directional impact by logical pattern, location as value, length of time in a given location, and said secondary or tertiary terms comprise at least one of a group comprising: impact in logical context, impact by relative context, location by logical pattern, location in logical context, location by relative context, body attitude, body attitude in logical context, body attitude in relative context, behavior pattern, behavior pattern in logical context, behavior pattern in relative context, audible sounds, audible sounds taken in logical context, audible sounds taken in relative context.

22. A method according to claim 3, wherein one of said secondary terms is impact and wherein said impact is processed at a firer level for categorization as one of a group comprising:
impact from behind; impact from in front; impact from the right; impact from the left; impact from above; impact from below; impact within a sequence; impact within a sequence within a time frame; impact as an absolute value; impact as a relative value; impact as part of a logical pattern; impact within a logical context, impact within a relative context.

23. The method of claim 1, wherein said measurements of primary body activities are obtained from at least one measurement unit located on the trunk of the body.

24. The method of claim 1, wherein said measurements include acoustic measurements or sound recording or radiation recording.

25. A personal emergency alarm network comprising:
at least on personal activity monitoring apparatus operative to perform at least one measurement of body activity,
an emergency monitoring server;
said personal activity monitoring apparatus operative to transmit said measurement to said emergency monitoring server;
said monitoring apparatus being operative to
provide at least one first nomenclature for at least one said measurement of a body activity surpassing said at least one threshold;
provide at least one second nomenclature for at least one a first combination of at least one of a concurrent combination, a sequential combination and a temporal combination, said first combination comprising at least one of said body activity and said first nomenclature;
provide at least one third nomenclature for at least one a second combination of at least one of a concurrent combination, a sequential combination and a temporal combination, said second combination comprising at least one of said body activity, said first nomenclature, said second nomenclature and said third nomenclature;
provide definitions of emergency situations;
associate said definitions of emergency with at least one of at least one of said body activity, said first nomenclature, said second nomenclature and said third nomenclature, and
send data comprising at least binary decision data resulting from said processing to said emergency monitor server.

26. The personal emergency alarm network of claim 25, wherein said at least binary decision data comprises:
i) the location of the event,
ii) occurrence of an event,
iii) the nature of the event.

27. The personal emergency alarm network of claim 26, wherein said occurrence, said location and said nature are sent respectively in order.

28. The personal emergency alarm network of claim 26, wherein said sending is arranged such as to firstly indicate a location of an event.
